(19) United States
(12) Patent Application Publication
(10) Pub. No.: US 2022/0370299 A1
(43) Pub. Date: Nov. 24, 2022

(54) Dental Opaquer Composition

(71) Applicant: Ivoclar Vivadent AG, Schaan (LI)

(72) Inventors: Corina Serban, Goldach (CH); Frank Rothbrust, Rons (AT); Christian Ritzberger, Grabs (CH); Sebastian Krolikowski, Lachen (CH); Vasiliki-Kallirroi Stoumari, Buchs (CH); Katja Ott, Eschen (LI); Christin Wildner, Mastrils (CH)

(21) Appl. No.: 17/664,101

(22) Filed: May 19, 2022

(30) Foreign Application Priority Data
May 20, 2021 (EP) 21175082.3

(51) Int. Cl. A61K 6/818; A61C 13/083; C03C 4/00; C03C 3/062; A61K 6/833; C03B 32/02

(52) U.S. Cl. CPC A61K 6/818; A61C 13/083; C03C 4/0021; C03C 3/062; A61K 6/833; C03B 32/02

(57) ABSTRACT

The invention relates to a dental opaquer composition which can be used to increase the opacity of zirconium oxide ceramics. In addition, the invention relates to a process for producing a dental restoration, in which the opacity of the zirconium oxide ceramic is increased by using the opaquer composition.

### CROSS-REFERENCE TO RELATED APPLICATIONS

This application claims priority to European Patent Application No. 21175082.3 filed on May 20, 2021, the disclosure of which is incorporated herein by reference in its entirety.

### TECHNICAL FIELD

The invention relates to a dental opaquer composition by which the opacity in dental oxide ceramics and in particular zirconium oxide ceramics can be increased.

### BACKGROUND

Zirconium oxide ceramics are widely used in the production of dental restorations due to their advantageous mechanical properties. Zirconium oxide materials based on tetragonal polycrystals stabilized with Y₂O₃ are particularly used as the starting material for dental restorations.

The production of dental restorations of zirconium oxide, as also described in WO 2018/115529 A1 and corresponding US 20190381769, which US published application is hereby incorporated by reference in its entirety, typically involve two thermal densification steps separated by a shaping step. In this process, the zirconium oxide starting materials are typically first cast or mechanically compacted using pressure and then presintered to an intermediate open-pored state to produce a blank. This blank is suitable for shaping or pre-shaping, for example by machining in a CAD/CAM process. The shaped blank can then be subjected to final thermal densification in a further sintering step.

Dental restorations not only have to meet high requirements in terms of mechanical properties. It is also of particular importance to imitate the complex external appearance of natural teeth in a lifelike manner. This involves imitating both translucency and coloration with the respective gradients and 3D effects. This is a particular challenge for dental restorations made of zirconium oxide, which is why other materials, such as lithium silicate glass ceramics, are preferred, especially in the area of anterior teeth, despite their often inferior mechanical properties to zirconium oxide.

It may occur in dental practice that a zirconium oxide restoration with high translucency is to be applied to a dark implant, such as a titanium framework, or a discolored prepared tooth stump. Due to the high translucency of the dental restoration, this may result in undesirable optical effects where the dark or discolored material is visible through the dental restoration. This problem occurs in particular where zirconium oxide restorations with thin walls are to be used, such as in cervical areas of dental restorations.

Various procedures are known to prevent dark materials from being visible through a zirconium oxide restoration.

For example, a framework of opaque zirconium oxide is first fabricated in complex procedures, which is then veneered with a glass ceramic and, if necessary, glazed.

It is also possible to reduce or completely prevent the visibility of a material located under the zirconium oxide ceramic with the help of dental opaquers such as infiltration solutions. For this purpose, the presintered dental restoration is brought into contact with the infiltration solution by applying the infiltration solution to the restoration or by immersing the restoration in the solution. The infiltration solution typically causes a white coloration and an increase in opacity in the dental restoration, which can prevent the dark materials from shining through the zirconium oxide restoration.

As a rule, the infiltration solutions are applied to the inside, i.e. into the lumen, of the dental restoration. The application of infiltration solutions to the outside of dental restorations is disadvantageous because undesirable interactions with colouring solutions applied or to be applied there may occur or the use of colouring solutions is completely prevented.

WO 2013/170705 A1 and corresponding U.S. Pat. No. 9,554,881, which US patent is hereby incorporated by reference in its entirety, disclose a colouring solution containing a combination of at least two types of colouring ions selected from the group consisting of the ions of Pr, Er, Ce and Nd, as well as solvent and an additive. When this colouring solution is applied to a zirconium oxide ceramic, the light transmission of the dental restoration increases, i.e. its opacity is reduced. The colouring solution is therefore not suitable for preventing dark materials from being visible through a dental zirconium oxide restoration.

EP 3 575 277 A1 and corresponding US 20190388196, which US published application is hereby incorporated by reference in its entirety, describes a liquid composition for increasing the opacity of a dental zirconium oxide ceramic. The opaquer composition contains a water-soluble aluminum or lanthanum compound, water and an organic solvent. However, it is known, for example, from EP 3 558 671 and corresponding US2019381769, which US published application is hereby incorporated by reference in its entirety, that doping with Al or La alters the local sintering properties. Therefore, it is desirable to avoid the use of Al- and La-containing opaquer compounds in order to ensure a low sintering distortion and thus a high accuracy of fit of the manufactured dental zirconium oxide ceramic.

WO 2019/146908 discloses a whitening agent composition, the application of which to zirconium oxide dental ceramics also leads to an increase in opacity. The whitening agent contains metal oxide nanoparticles and/or a metal ion-containing component as well as a stabilizing agent. However, the use of metal oxide nanoparticles is costly, as it requires multi-stage processing, and also poses high health risks for the user due to the use of hydrofluoric acid.

WO 2015/148215 A1 and corresponding U.S. Ser. No. 10/350,032, which US patent is hereby incorporated by reference in its entirety, disclose a phosphorus-containing composition for increasing the opacity of zirconium oxide ceramics. However, it is known that the treatment of zirconium oxide materials with phosphorus-containing compositions, such as phosphorus-containing acids, is disadvantageous because this has a negative influence on the bond to adhesives, such as to phosphate-containing cements. Therefore, in order to ensure a strong bond between the inner surface of the dental restoration and the tooth stump, the application of phosphate-containing compositions to the inner surface of the dental restoration, i.e. in its lumen, should be avoided as far as possible. However, it is precisely there that the opacifying effect would be desirable in order to reduce the visibility of discolored tooth stumps.

### SUMMARY

The invention is based on the problem of providing an agent by which the opacity of zirconium oxide ceramics can be increased. A zirconium oxide ceramic treated with this agent should not only exhibit advantageous properties in terms of esthetic appearance, but also in terms of mechanical properties and accuracy of fit. The agent should also allow use in thin-walled dental restorations. Furthermore, the agent should be harmless to health and allow uncomplicated processing. In addition, the agent should have a high stability and long-lasting homogeneity during storage.

This problem is surprisingly solved by the dental opaquer composition according to the claims. The invention also relates to the process of producing a dental restoration according to the claims.

### BRIEF DESCRIPTION OF THE DRAWINGS

The invention in the following will be described in detail by referring to exemplary embodiments that are accompanied by figures, in which:

FIG. 1 shows densely sintered zirconium oxide ceramics;

FIG. 2 shows presintered zirconium oxide ceramics treated with an opaquer composition;

FIG. 3 shows sintered zirconium oxide ceramics treated with an opaquer composition;

FIG. 4 shows the microstructure of a surface area treated with an opaquer composition; and

FIG. 5 shows a specimen of a 3D measurement using an optical scanner.

### DETAILED DESCRIPTION

The dental opaquer composition according to the invention is characterized in that it comprises
(a) alkali silicate and
(b) water,

wherein the alkali silicate is at least partially dissolved in the water.

Surprisingly, it has been found that the composition according to the invention can be used to effect a high opacity in oxide ceramics, in particular zirconium oxide ceramics, even without the use of opaquing metal ions, such as ions of Al, La, Zr and/or Ti, or metal oxide nanoparticles containing, for example, Al, Ti and/or Zr.

The opaquing effect of the composition can be limited to areas of the oxide ceramic near the surface, making it possible to achieve the desired esthetic effects for covering discolored tooth stumps or dark implants even in thin-walled highly esthetic dental restorations.

Furthermore, the composition according to the invention surprisingly enables dental restorations to be produced which, in addition to the desired esthetic properties, also exhibit particularly advantageous mechanical properties. Dental restorations with the desired opacity properties can be obtained, which are characterized by strength and long-term stability in the range of oxide ceramics, in particular zirconium oxide ceramics, which have not been treated with the opaquer composition.

Furthermore, the composition according to the invention can also be used to obtain dental restorations that are characterized not only by the desired opacity properties but also by a high accuracy of fit. Oxide ceramics treated with the composition exhibit an advantageous sintering behavior with low sintering distortion during final dense sintering. Dental restorations with the desired esthetic properties can therefore be obtained, which can also be placed in the patient's mouth without an additional step to adjust the fit to the prepared stump or implant interface.

For the purposes of the present invention, the term “opaquer composition” refers to a composition which is suitable for increasing the opacity of an oxide ceramic, in particular a zirconium oxide ceramic. In this context, the increase in opacity occurs in particular after the oxide ceramic has been brought into contact with the composition and additionally subjected to a heat treatment. The increase in opacity occurs in particular in the oxide ceramic itself and not by applying an opaque layer to the oxide ceramic. The opaquer composition is particularly suitable for increasing the opacity in a presintered zirconium oxide ceramic, which is preferably made of tetragonal polycrystals stabilized with Y₂O₃.

Opacity is the impermeability to light and is reciprocal to translucency, i.e. the light transmission of a material. Translucency is the ratio of transmitted to incident light intensity. As a measure of opacity or translucency, the contrast value (CR value) can be determined in accordance with British Standard 5612. An increase in opacity according to the invention is present if in a part of an oxide ceramic, in particular zirconium oxide ceramic, such as a densely sintered dental restoration, which has been treated with the opaquer composition and optionally heat-treated, the opacity is increased compared to another untreated part of the oxide ceramic. If the entire oxide ceramic has been treated with the composition, an untreated oxide ceramic prepared in a corresponding manner can be used to determine the comparative value. The increase in opacity is also referred to as “opacifying” for the purposes of the present application.

For the purposes of the invention, the terms “color” and “colored” refer to the color value and/or brightness of a material. Color values and brightnesses can be determined by the L*a*b value, in particular according to DIN EN ISO 11664-4, or according to a shade guide commonly used in the dental industry. The color measurement can be performed with commercially available measuring instruments, such as a CM-3700d spectrophotometer (Konica Minolta). Examples of shade guides are the Vitapan Classical® and the Vita 3D Master®, both from VITA Zahnfabrik H. Rauter GmbH & Co. KG, and the Chromascop® of Ivoclar Vivadent AG.

Without limitation to any theory, it is assumed that thin glass phases can form on the oxide ceramic grains in the oxide ceramic after the opaquer composition has been applied. It is assumed that the increase in opacity is due to the difference in refractive indices of oxide ceramic and glass phases.

It is preferred that the alkali silicate is selected from lithium silicates, sodium silicates, potassium silicates and mixtures thereof, and in particular sodium silicates.

Preferably, the alkali silicate may be in the form of sodium water glass, potassium water glass or lithium water glass and, in particular, sodium water glass. For the purposes of the present invention, sodium water glass is water glass comprising sodium silicates and optionally other alkali silicates, wherein sodium silicates represent the largest proportion by weight of the silicates. Accordingly, potassium water glass and lithium water glass have a predominant proportion by weight of potassium silicates and lithium silicates, respectively. Preferably, the density of the sodium water glass is 1.34 to 1.38 g/cm³, the density of the potassium water glass is 1.23 to 1.27 g/cm³, in particular 1.25 g/cm³, and the density of the lithium water glass is 1.16 to 1.20 g/cm³, in particular 1.18 g/cm³.

In a preferred embodiment, the composition comprises 0.5 to 37 wt.-%, preferably 0.5 to 20 wt.-%, more preferably 1 to 10 wt.-% alkali silicate.

Alkali silicates present in the opaquer composition can be qualitatively detected or quantitatively determined by, for example, X-ray fluorescence analysis (XRF), Si-NMR, ¹³C-NMR, atomic absorption spectrometry (AAS) or Raman spectroscopy, optionally in combination with further indirect analyses such as pH measurements, residue on ignition analyses or scanning electron microscopic analyses of dried or crystallized opaquer composition.

In a further preferred embodiment, the composition comprises 0.001 to 10 wt.-%, preferably 0.001 to 8 wt.-%, in particular 0.001 to 5 wt.-% Si, calculated as SiO₂, and 0.0001 to 10 wt.-%, preferably 0.001 to 8 wt.-%, in particular 0.01 to 5 wt.-%, alkali metal, calculated as alkali metal oxide.

It is further preferred that the composition comprises 63 to 99.5 wt.-%, preferably 80 to 99.5 wt.-%, more preferably 90 to 99.0 wt.-% water.

In a further preferred embodiment, the weight ratio of alkali silicate to water in the composition is in the range of 1:200 to 1:6, in particular 1:100 to 1:8.

It is further preferred that the alkali silicate is completely dissolved in the water.

In a further preferred embodiment, the alkali silicate is present in the form of water glass, wherein the weight ratio of Si, calculated as SiO₂, to alkali metal, calculated as alkali metal oxide, is 1 to 6, preferably 1.5 to 6 and particularly preferably 2 to 6.

In another preferred embodiment, the composition according to the invention comprises a thickening agent, preferably an organic thickening agent.

A thickening agent is understood to be an agent by which the viscosity of a solution, preferably an aqueous solution, can be increased.

It was found that the addition of a thickener can influence how deeply the opaquer composition penetrates into the zirconium oxide ceramic. This is particularly advantageous if the opacifying effect of the composition is to be limited to areas near the surface. In this way, coverage of discolored stumps or dark implants can be achieved even in thin-walled dental restorations, which is not accompanied by undesirable esthetic effects on the outside of the restoration.

In a preferred embodiment, the thickening agent is a polysaccharide. It is further preferred that the thickening agent is selected from the group consisting of polyvinylpyrrolidone, polyethylene glycol, polypropylene glycol, hydroxyethyl cellulose, ethyl cellulose, methyl cellulose, xanthan gum, carbomer, pectins, silica sol, starch, gelatin, alginates, and mixtures thereof, and particularly preferably is polyvinylpyrrolidone, polyethylene glycol, or a mixture thereof. Particularly preferably, the thickening agent is polyvinylpyrrolidone.

It is preferred that the polyvinylpyrrolidone has a molecular weight in the range of 10,000 to 1,000,000 g/mol and the polyethylene glycol has a molecular weight in the range of 200 to 100,000 g/mol, based in each case on the weight average molecular weight.

Polyvinylpyrrolidone with a weight average molecular weight of 10,000 to 500,000 g/mol, in particular 10,000 to 100,000 g/mol, is particularly preferred.

It was found that the use of polyvinylpyrrolidone markedly facilitates the preparation of clear solutions of the composition according to the invention. In particular, using polyvinylpyrrolidone, it is possible to prepare the compositions more quickly, to obtain clear solutions and also to omit extensive dissolving and mixing procedures, such as the use of an ultrasonic bath.

The use of polyvinylpyrrolidone has also proven to be advantageous with respect to the stability of the composition according to the invention. Adverse storage effects of the composition, such as phase separation or precipitation, can be avoided by the use of polyvinylpyrrolidone.

In particular, it has been shown that a composition containing polyvinylpyrrolidone is less prone to the formation of undesirable turbidity and flocculation compared to compositions containing other thickening agents. Since such turbidity and flocculation is visible to the naked eye, their formation can be monitored, for example, by observing the composition.

Polyethylene glycol with a weight average molecular weight of 200 to 50,000 g/mol, in particular 200 to 35,000 g/mol, is particularly preferred.

It is preferred that the composition comprises 0 to 50 wt.-%, preferably 0.1 to 50 wt.-%, in particular 0.2 to 30 wt.-%, particularly preferably 0.5 to 20 wt.-% thickening agent.

In a preferred embodiment, the composition comprises 0 to 30 wt.-%, preferably 0.1 to 30 wt.-%, in particular 0.2 to 20 wt.-%, particularly preferably 0.5 to 10 wt.-%, even further preferred 0.5 to 6 wt.-% polyvinylpyrrolidone.

In a further preferred embodiment, the weight ratio of thickening agent, in particular polyvinylpyrrolidone, to water in the composition is in the range of 1:200 to 1:10, in particular 1:100 to 1:12, particularly preferably 1:50 to 1:15.

It is also preferred that the weight ratio of thickening agent, in particular polyvinylpyrrolidone, to alkali silicate in the composition is in the range of 1:20 to 5:1, in particular 1:15 to 1:4.

In another preferred embodiment, the composition comprises 0 to 50 wt.-%, preferably 0.1 to 50 wt.-%, in particular 0.2 to 30 wt.-%, particularly preferably 0.5 to 20 wt.-% polyethylene glycol-.

It has been shown that the penetration depth of the opaquer composition can be influenced by the proportion of thickening agent, such as polyvinyl pyrrolidone and polyethylene glycol, as well as the properties of the thickening agent. As a rule, for example, a high proportion of polyvinyl pyrrolidone or polyethylene glycol, as well as a high molecular weight of these components, leads to a low penetration depth, whereby the opacifying effect can be decreased for the observer.

In a preferred embodiment, the opaquer composition according to the invention further comprises an organic solvent, preferably in an amount of 0.5 to 30 wt.-%, in particular 1 to 20 wt.-%. Suitable organic solvents are in particular miscible with water and selected from the group consisting of methanol, ethanol, isopropanol, glycerol, ethylene glycol, acetone, 1,4-dioxane and mixtures thereof. Advantageous wetting and drying properties of the opaquer composition can result from the addition of organic solvents, for example.

In a preferred embodiment, the composition is substantially free of metal oxide nanoparticles. It is further preferred that the composition is substantially free of Al, La, Zr, P and Ti.

It is further preferred that the opaquer composition comprises an identification color. For the purposes of the present invention, an identification color is an agent which has a coloring effect in the composition without having a significant influence on the color of the densely sintered oxide ceramic, in particular zirconium oxide ceramic. Preferred identification colors are organic dyes, in particular triphenylmethane dyes, such as patent blue V (E131). The identification color can improve the visibility of the composition according to the invention on the presintered oxide ceramic and thereby facilitate the precise application of the composition.

It is further preferred that the composition according to the invention is in the form of a liquid and in particular in the form of a solution. This ensures easy application to the oxide ceramic, e.g. with the aid of a brush or a suitable applicator. Particularly preferably, the composition is in the form of a homogenous liquid to ensure a uniform opacifying effect.

It is particularly preferred that the composition has a pH value in the range of 7.5 to 12.5, preferably 8.5 to 12.5, particularly preferably 9.5 to 12. In this way, the risk of acid or alkaline burns for users of the opaquer composition is reduced. In particular, there is an improvement in occupational safety for dental technicians and dentists compared to the opaquer compositions known from the prior art with a pH value above 12.5.

In another preferred embodiment, the composition has a viscosity at room temperature, i.e. at a temperature of 23° C., in the range of 1 to 5,000, preferably 1 to 1,000, particularly preferably 1 to 100 mPa*s in the shear rate range of 1 to 100 s⁻¹. The viscosity can be determined, for example, with a rheometer of type MCR302 from Anton Paar GmbH and using a CP50 measuring system (cone plate with 50 mm diameter). Typically, compositions with a viscosity within the ranges mentioned have particularly advantageous properties in terms of processability.

The invention further relates to a process for producing a dental restoration, in which the opacity is increased in at least a part of an oxide ceramic, in particular a zirconium oxide ceramic, by applying a composition according to the invention described above to this part and subjecting it to a heat treatment.

Preferably, the heat treatment is carried out for at least 1 min, in particular at least 10 min at a temperature of preferably 1000 to 1700° C., in particular 1000° C. to 1600° C., more preferably 1000 to 1500° C.

It is preferred that the composition penetrates into the oxide ceramic to a depth of 1 to 2000 μm, in particular 2 to 1000 μm and particularly preferably 5 to 500 μm. The penetration depth can be determined by treating the oxide ceramic with the composition, subjecting it to heat treatment and then dividing it, for example using a saw, and afterwards measuring how far the composition has penetrated into the oxide ceramic on the surface, which has been made accessible by the dividing, using a microscope (e.g. Olympus SZX10).

It is also preferred to apply the opaquer composition in the lumen of the dental restoration to be fabricated.

In a preferred embodiment, the opacity is increased in at least a part of an oxide ceramic to which a barrier agent has been previously applied and optionally dried. By a barrier agent is meant an agent which, after application and optionally drying to an oxide ceramic, can reduce the depth of penetration of the opaquer composition in the oxide ceramic. As a rule, the barrier agent forms a barrier layer in the oxide ceramic which counteracts the penetration of the opaquer composition.

In a preferred embodiment, the barrier agent comprises a solvent, in particular an organic solvent, and a polymer dispersed therein, in particular a thickening agent, and optionally an identification color. Suitable thickening agents are those mentioned for the opaquer composition.

In another preferred embodiment, the same composition or a different composition according to the invention is applied again in at least a region of the part of the oxide ceramic to which the opaquer composition has been applied. Preferably, prior to repeated application, the composition is first dried before the same or a different composition according to the invention is applied again. By applying the opaquer composition multiple times, optionally in combination with selecting compositions with different properties, such as different penetration depths, it is also possible to generate complicated three-dimensional opacity gradients.

The oxide ceramic, in particular zirconium oxide ceramic, can be in an unsintered or presintered state when the composition according to the invention is applied. It is particularly preferred that the oxide ceramic is presintered. The presintered oxide ceramic is typically present in an open-pore state with a density of preferably 30 to 70%, in particular 40 to 65%, of the density of the densely sintered oxide ceramic. Typically, a lower density of the presintered oxide ceramic is accompanied by a greater penetration depth of the opaquer composition.

In a particularly preferred embodiment, the heat treatment is accompanied by the dense sintering of the oxide ceramic.

Typically, dense sintering results in a dental restoration with the desired properties. It has been shown that oxide ceramics, in particular zirconium oxide ceramics, in which the opacity has been increased by means of the composition according to the invention, exhibit an advantageous sintering behavior with low sintering distortion.

The process according to the invention allows dental restorations with advantageous mechanical properties to be produced.

Preferably, a densely sintered dental restoration produced according to the process of the invention has a biaxial fracture strength of at least 500 MPa, in particular at least 600 MPa and particularly preferably at least 700 MPa, determined according to ISO 6872:2015.

It is also preferred that a densely sintered dental restoration produced according to the process of the invention has a biaxial fracture strength of at least 70%, preferably at least 80%, in particular at least 95%, determined according to ISO 6872:2015 and based in each case on the biaxial fracture strength of a corresponding densely sintered dental restoration that has not been treated with the opaquer composition.

It is also preferred that a densely sintered dental restoration prepared according to the process of the invention exhibits a high long-term stability. Particularly preferably, the densely sintered dental restoration is characterized by passing a fatigue test in which the dental restoration is loaded for 2,000,000 test cycles with at least 450 N, preferably at least 550 N, particularly preferably at least 650 N, using a 5 kN servopneumatic two-column table-top testing machine from DYNA-MESS Prüfsysteme GmbH with a sinusoidal frequency (preload is 10% of the main load) of 5 Hz in 37° C. warm water.

Dental restorations produced according to the process of the invention can be attached to a stump or implant framework by any known method, e.g. cementing, bonding or screwing.

Chemical and mechanical methods for pretreating the surface of zirconium oxide to strengthen the bond to the stump or implant surface are known from the prior art.

In a preferred embodiment of the process, treatment with an etching liquid is carried out in the part of the oxide ceramic in which the opacity of the oxide ceramic has been increased. The treatment may occur in a portion of the opacified part and may extend to non-opacified parts of the oxide ceramic. Surprisingly, it has been shown that treatment with an etching liquid can also lead to an improvement in the bond with the stump or implant in the areas treated with the opaquer composition.

In a particularly preferred embodiment, the etching liquid comprises hydrofluoric acid.

In another preferred embodiment of the process, the dental restoration is a bridge, an inlay, an onlay, a veneer, an abutment, a partial crown, a crown or a facet.

The invention further relates to the use of the dental opaquer composition according to the invention for increasing the opacity in an oxide ceramic, in particular zirconium oxide ceramic, in the production of a dental restoration.

All compositional definitions, process steps and process parameters described in the context of the opaquer composition and process according to the invention are also suitable for this use according to the invention.

The invention is explained in more detail below with reference to examples.

### EXAMPLES

### Examples 1 and 2: Opaquer Compositions According to the Invention

Solutions were prepared with the chemical compositions according to Table 1.

TABLE 1

Example

| | 1 | 2 |
|---|---|---|
| Component | Weight (g) | Weight (g) |
| H₂O | 21.37 | 20.00 |
| Polyvinylpyrrolidone K30 | 1.125 | 1.00 |
| Sodium water glass | 2.55 | 1.05 |
| (33.6 to 37 wt.-% solution) | | |

Sodium water glass of the purity class “ultrapure” from Carl Roth GmbH, which according to the manufacturer does not contain any other alkali silicates apart from sodium silicate, and polyvinylpyrrolidone (PVP) K30 of the purity class “ultrapure” from BASF SE, which is sold under the name Luviskol® K30, were used for preparing these and the following examples. According to the manufacturer, Luviskol® K30 consists of polyvinylpyrrolidone with a K-value (according to Fikentscher, 1% in water) between 27.0 and 33.0 and a weight average molecular weight of about 49,000 g/mol. The content in wt.-% given in Table 1 and all subsequent tables and compositions for the respective components corresponds to the manufacturer's specification.

Two presintered zirconium oxide ceramics in the form of dental crowns were provided. The solutions of Examples 1 and 2 were applied with a brush to the inner surface of each crown and air dried. Subsequently, the crowns were densely sintered for 120 min at 1500° C. in a Programat S1-1600 furnace of Ivoclar Vivadent AG.

After dense sintering, opacity was observed in both crowns in the surface areas treated with the solutions and was increased compared to the areas not treated with the solutions.

### Examples 3 to 5: Opaquer Compositions with PVP K30, K90 and K360

Opaquer compositions were prepared with the chemical compositions according to Table 2.

TABLE 2

| | Example | | |
|---|---|---|---|
| | 3 | 4 | 5 |
| Component | Weight (g) | Weight (g) | Weight (g) |
| H₂O | 21.37 | 21.37 | 21.37 |
| Polyvinylpyrrolidone K30 | 1.125 | — | — |
| Polyvinylpyrrolidone K90 | — | 1.125 | — |
| Polyvinylpyrrolidone K360 | — | — | 1.125 |
| Sodium water glass | 2.5 | 2.5 | 2.5 |
| (33.6 to 37 wt.-% solution) | | | |

Three presintered zirconium oxide ceramics were provided in the form of dental crowns, then treated with the opaquer compositions of Examples 3 to 5 and subjected to heat treatment according to the procedure described for Examples 1 and 2.

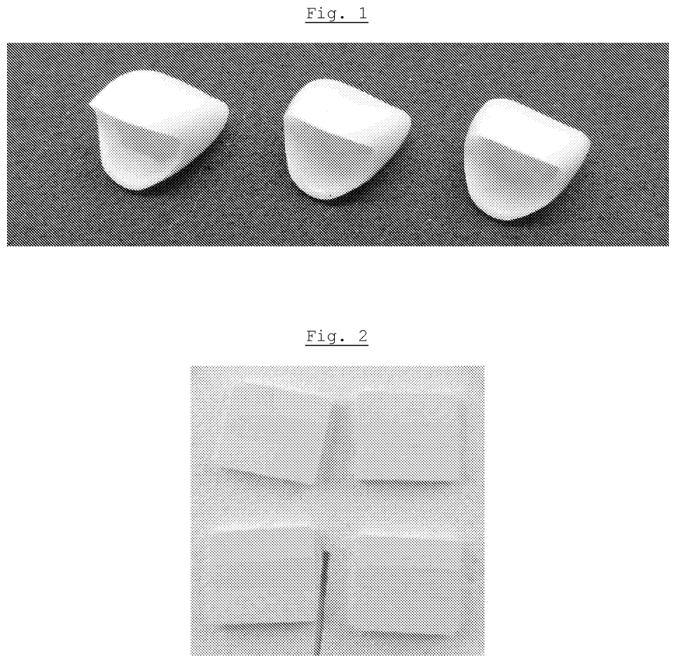

The densely sintered zirconium oxide ceramics are shown in FIG. 1, where the zirconium oxide ceramic on the left has been treated with the opaquer composition of Example 3, the zirconium oxide ceramic in the center with the opaquer composition of Example 4 and the zirconium oxide ceramic on the right with the opaquer composition of Example 5. It can be seen that for the observer, the opacifying effect of the composition according to Example 3, i.e. using polyvinylpyrrolidone K30, is the strongest.

### Example 6: Opaquer Compositions with Identification Color

A solution with the chemical composition according to Table 3 was prepared and applied in up to four layers to platelet-shaped presintered zirconium oxide ceramics.

TABLE 3

| Component | Weight (g) |
|---|---|
| H₂O | 20 |
| Polyvinylpyrrolidone K30 | 1 |
| Sodium water glass | 1.05 |
| (33.6 to 37 wt.-% solution) | |
| Patent blue V (E131) | <0.01 |

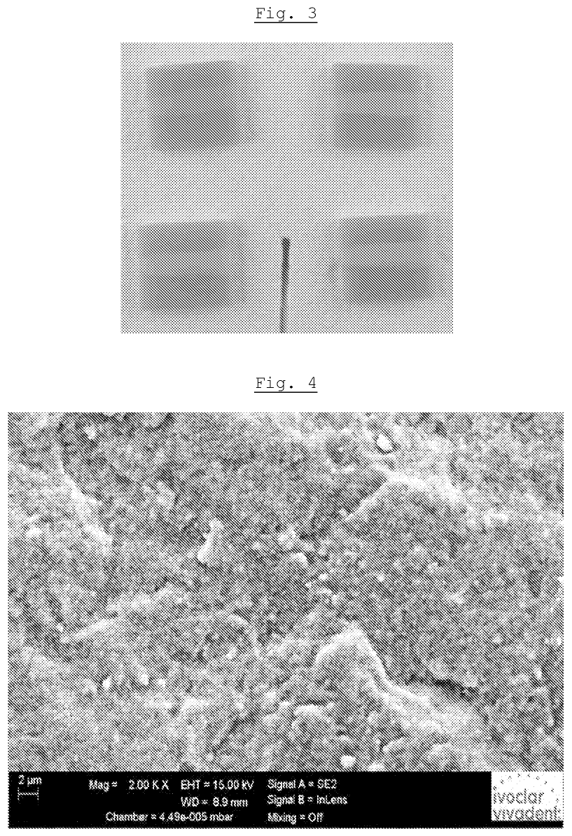

The presintered zirconium oxide ceramics treated with the opaquer composition are shown in FIG. 2. Due to the addition of the identification color, the areas of the zirconium oxide ceramics treated with the opaquer composition were clearly distinguishable from the untreated areas.

The zirconium oxide ceramics were densely sintered according to the procedure described for Examples 1 and 2. The opacity of the densely sintered zirconium oxide ceramics was increased in the areas which had been treated with the opaquer composition compared to the untreated areas. This is also evident from the zirconium oxide ceramics shown in FIG. 3.

### Example 7: Determination of the Penetration Depth of Opaquer Solutions

A platelet-shaped presintered zirconium oxide ceramic was provided and treated with the opaquer solution with the chemical composition according to Table 4 by applying about 3 μl of opaquer solution to the zirconium oxide ceramic.

TABLE 4

| Component | Weight (g) |
|---|---|
| H₂O | 9.9 |
| Sodium water glass | 0.1 |
| (33.6 to 37 wt.-% solution) | |

The zirconium oxide ceramic was densely sintered according to the procedure described for Examples 1 and 2 and then cut in order to be able to determine the penetration depth of the opaquer solution. The measurement using an Olympus SZX10 microscope resulted in a penetration depth of 0.14 mm.

### Example 8: Analysis of the Microstructure by SEM

A presintered zirconium oxide ceramic was treated with the opaquer composition according to Table 5.

TABLE 5

| Component | Weight (g) |
|---|---|
| H₂O | 9 |
| Sodium water glass | 1 |
| (33.6 to 37 wt.-% solution) | |

The zirconium oxide ceramic was densely sintered according to the procedure described for Examples 1 and 2. Subsequently, the microstructure of the zirconium oxide ceramic was examined by scanning electron microscopy (SEM). The microstructure of a surface area treated with the composition according to Table 5 is shown in FIG. 4. It can be seen that the structure of the zirconium oxide ceramic is characterized by a small grain size. Furthermore, no pores are visible.

### Examples 9 to 11: Test Specimens for Determining the Biaxial Fracture Strength

To determine the biaxial fracture strength according to ISO 6872:2015, disc-shaped test specimens with a thickness of 1.2 mm and a diameter of 13.0 mm were fabricated from presintered zirconium oxide.

A barrier agent containing 65.37 wt.-% H₂O, 18.247 wt.-% PVP K30, 16.343 wt-.% ethanol and 0.04 wt.-% patent blue V (E131) was applied to the test specimens of Examples 10 and 11 and dried. Three opaquer solutions were prepared with chemical compositions according to Table 6.

TABLE 6

| | Example | | |
|---|---|---|---|
| | 9 | 10 | 11 |
| Component | Weight (g) | Weight (g) | Weight (g) |
| H₂O | 9.9 | 9.5 | 9 |
| Sodium water glass | 0.1 | 0.5 | 1 |
| (33.6 to 37 wt.-% solution) | | | |

Five test specimens each were treated with the opaquer compositions of Examples 9 to 11 by applying 3 μl of opaquer composition to each test specimen. All test specimens were densely sintered, placed on three concentrically arranged balls and loaded centrally until failure.

The average biaxial fracture strength of the test specimens of Examples 9 to 11 is shown in Table 7.

TABLE 7

| | Mean biaxial fracture |
|---|---|
| Example | strength (MPa) |
| 9 | 988 |
| 10 | 947 |
| 11 | 899 |

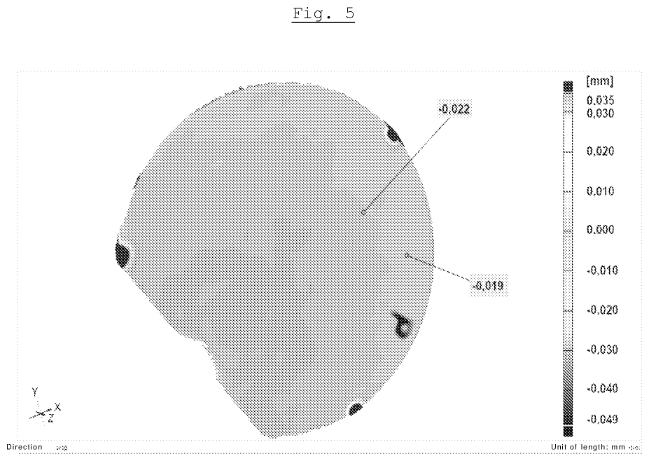

No deformation was visible on the test specimens of examples 9 to 11 after dense sintering. For illustration purposes, the specimens were subjected to a 3D measurement using an optical 3D scanner (model ATOS Capsule from GOM GmbH). An exemplary result of the 3D measurement is shown in FIG. 5.

### Example 12: Fatigue Test

Four 3-unit posterior bridges were fabricated from zirconium oxide (e.max ZirCAD from Ivoclar Vivadent AG). The bridges had a wall thickness of 0.6 mm and connector cross-sections of about 3 mm×3 mm.

An opaquer composition according to the invention was prepared from 21.37 g H₂O, 1.125 g PVP K30 and 2.5 g sodium water glass (33.6 to 37 wt.-% solution). The composition was applied to the entire luminal surface of two posterior bridges. No opaquer composition was applied to the surface of the two remaining posterior bridges. All four posterior bridges were densely sintered.

The posterior bridges were then attached to polymethyl methacrylate (PMMA) stumps by first mechanically roughening the inner surface of the posterior bridges and the upper surfaces of the stumps by sandblasting and then bonding them together with a self-curing glass ionomer cement (Vivaglass CEM from Ivoclar Vivadent AG).

The posterior bridges were then subjected to a fatigue test in a servo-pneumatic two-column table-top testing machine 5 kN from DYNA-MESS Prüfsysteme GmbH (two-column table-top testing machine 5 kN), in which the posterior bridges were loaded for 2,000,000 cycles with 550 N at a sinusoidal frequency (preload is 10% of the main load) of 5 Hz in 37° C. warm water.

Both the two posterior bridges treated with the opaquer composition and the two bridges not treated with the opaquer composition showed no visible damage after completion of the fatigue test.

### Examples 13 and 14: Homogeneity of the Composition

Two opaquer compositions were prepared with the chemical compositions according to Table 8. For this purpose, the sodium water glass was first homogenized with the water for about 15 min at room temperature. Then the thickening agent was added and another homogenisation step was carried out. A magnetic stirrer with a speed of 400 to 600 rpm was used for homogenisation.

TABLE 8

Example

| | 13 | 14 |
|---|---|---|
| Component | Weight (g) | Weight (g) |
| H₂O | 21.37 | 21.37 |
| Polyvinylpyrrolidone K30 | 1.125 | — |
| Polyethylene glycol 600 | — | 1.125 |
| Sodium water glass | 2.5 | 2.5 |
| (33.6 to 37 wt.-% solution) | | |

The compositions were closely inspected for homogeneity immediately after their preparation. The compositions were then stored at room temperature and regularly reinspected for homogeneity over a period of several weeks.

For the composition of Example 14, flocculation was observed immediately after its preparation, and the amount of the formed floccules increased visibly as the storage time progressed. The floccules settled on the bottom of the vial.

The composition of Example 13, on the other hand, was clear immediately after production and no flocculation was observed. The first floccules were only visible after more than two weeks and flocculation was considerably slower than in the composition of Example 14.

1. A dental opaquer composition, which comprises
(a) alkali silicate and
(b) water,
wherein the alkali silicate is at least partially dissolved in the water.

2. The composition according to claim 1, which comprises a thickening agent.

3. The composition according to claim 2, wherein the thickening agent is selected from the group consisting of polyvinylpyrrolidone, polyethylene glycol, polypropylene glycol, hydroxyethyl cellulose, ethyl cellulose, methyl cellulose, xanthan gum, carbomer, pectins, silica sol, starch, gelatin, alginates, and mixtures thereof.

4. The composition according to claim 1, wherein the alkali silicate is selected from lithium silicates, sodium silicates, potassium silicates and mixtures thereof.

5. The composition according to claim 3, wherein the polyvinylpyrrolidone has a molecular weight in the range of 10,000 to 1,000,000 g/mol and the polyethylene glycol has a molecular weight in the range of 200 to 100,000 g/mol, each based on the weight average molecular weight.

6. The composition according to claim 1, which comprises 0.5 to 37 wt.-% alkali silicate.

7. The composition according to claim 1, which comprises 63 to 99.5 wt.-% water.

8. The composition according to claim 2, which comprises 0 to 50 wt.-% thickening agent.

9. The composition according to claim 3, which comprises 0 to 30 wt.-% polyvinylpyrrolidone.

10. The composition according to claim 3, which comprises 0 to 50 wt.-% polyethylene glycol.

11. The composition according to claim 1, which is present in the form of a liquid.

12. The composition according to claim 11, which has a pH value in the range of 7.5 to 12.5.

13. The composition according to claim 1, which has a viscosity in the range of 1 to 5,000 mPa*s in the shear rate range of 1 to 100 s⁻¹.

14. A process for producing a dental restoration, wherein the opacity is increased in at least a part of an oxide ceramic, by applying a composition according to claim 1 to the part and subjecting the part to a heat treatment.

15. The process according to claim 14, wherein the oxide ceramic is presintered.

16. The process according to claim 14, wherein the heat treatment is accompanied by a dense sintering of the oxide ceramic.

17. The process according to claim 14, wherein treatment with an etching liquid is carried out in the part of the oxide ceramic in which the opacity has been increased.

18. The process according to claim 14, wherein the dental restoration is a bridge, an inlay, an onlay, a veneer, an abutment, a partial crown, a crown or a facet.

19. A process of using the composition according to claim 1, for increasing the opacity in an oxide ceramic in the production of a dental restoration comprising applying the composition to the oxide ceramic, which oxide ceramic is unsintered or presintered.
